(19) United States
(12) Patent Application Publication
(10) Pub. No.: US 2008/0221163 A1
(43) Pub. Date: Sep. 11, 2008

(54) Selective Estrogen Receptor Modulators

(76) Inventors: Jeffrey Alan Dodge, Indianapolis, IN (US); Randall Bruce Hopkins, Indianapolis, IN (US); Owen Brendan Wallace, Zionsville, IN (US)

Correspondence Address: ELI LILLY & COMPANY, PATENT DIVISION, P.O. BOX 6288, INDIANAPOLIS, IN, 46206-6288, US

(21) Appl. No.: 10/597,090

(22) PCT Filed: Jan. 18, 2005
(86) PCT No.: PCT/US05/00019

§ 371 Date: Jul. 11, 2006

(51) Int. Cl. A61K 31/453; C07D 407/12; A61P 35/00

(52) U.S. Cl. 514/320; 546/196

(57) ABSTRACT

The present invention relates to a selective estrogen receptor modulator of formula I: I; or a pharmaceutical acid addition salt thereof; useful, e.g., for treating endometriosis and uterine leiomyoma.

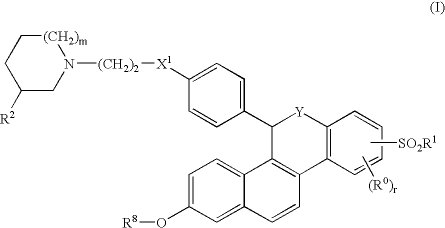

### FIELD OF INVENTION

The present invention is in the field of medicine, particularly in the treatment of gynecological disorders. More specifically, the present invention relates to selective estrogen receptor modulators useful to treat endometriosis and uterine leiomyoma.

### BACKGROUND OF THE INVENTION

Uterine leiomyoma/leiomyomata (uterine fibroid disease) is an old and ever present clinical problem that goes under a variety of names, including uterine fibrosis, uterine hypertrophy, uterine leiomyomata, symmetrical hypertrophy, fibrosis uteri, and fibrotic metritis. Essentially, uterine fibrosis is a condition where there is an inappropriate deposition of fibroid tissue on the wall of the uterus. This condition is a cause of dysmenorrhea and infertility in women.

Endometriosis is a condition of severe dysmenorrhea, which is accompanied by severe pain, bleeding into the endometrial masses or peritoneal cavity and often leads to infertility. The symptom's cause appears to be ectopic endometrial growths that respond inappropriately to normal hormonal control and are located in inappropriate tissues. Because of the inappropriate locations for endometrial growth, the tissue seems to initiate local inflammatory-like responses causing macrophage infiltration and a cascade of events leading to initiation of the painful response. Evidence suggests that a cause of uterine fibrosis and endometriosis is an inappropriate response of fibroid tissue and/or endometrial tissue to estrogen.

Many publications have appeared within the last ten years disclosing novel selective estrogen receptor modulators (SERMs), e.g., U.S. Pat. Nos. 5,484,795, 5,484,798, 5,510,358, 5,998,401 and WO 96/09040. Many of these SERMs, generally speaking, have been found to have a beneficial estrogen agonist activity in the bone and cardiovascular systems with a concomitant beneficial estrogen antagonist activity in the breast. A small, particularly useful subset of such compounds has also been found to have an estrogen antagonist effect in the uterus. A compound with this particularly useful SERM profile holds particular promise in treating uterine leiomyoma/leiomyomata and/or endometriosis.

However, the actual use of these SERM compounds, particularly in pre-menopausal women, has been hampered due to said compound's stimulatory effect on the ovaries. A great need currently exists, therefore, for new SERM compounds that behave as estrogen antagonists in the uterus that do not stimulate the ovaries.

### SUMMARY OF INVENTION

The present invention relates to a compound of formula I:

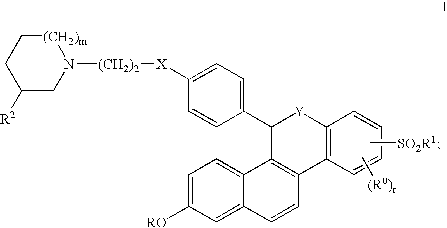

wherein:

m and r are independently 0, 1 or 2;

R is H, SO₂(n-C₄-C₆ alkyl) or COR³;

R⁰ is independently at each occurrence OH, CF₃, halo, C₁-C₆ alkyl or C₁-C₆ alkoxy;

R¹ is C₁-C₆ alkyl, C₁-C₆ alkoxy, NR⁴R^{4a}, CF₃ or CH₂CF₃;

R² is H or methyl provided that if m is 1 or 2, then R² must be H and that if m is 0, then R² must be methyl;

R³ is C₁-C₆ alkyl, C₁-C₆ alkoxy, NR⁶R^{6a}, phenoxy, or phenyl optionally substituted with halo;

R⁴ is C₁-C₆ alkyl or phenyl;

R^{4a}, R⁶ and R^{6a} are independently H, C₁-C₆ alkyl or phenyl;

X is O or NR⁷;

Y is O or S; and

R⁷ is H or C₁-C₆ alkyl; or a pharmaceutical acid addition salt thereof.

The present invention also relates to a pharmaceutical composition that comprises a compound of formula I, or a pharmaceutical acid addition salt thereof, and a pharmaceutical carrier. In another embodiment, the pharmaceutical composition of the present invention may be adapted for use in treating endometriosis and/or uterine leiomyoma.

The present invention also relates to methods for treating endometriosis and/or uterine leiomyoma employing a compound of formula I, or a pharmaceutical acid addition salt thereof.

In addition, the present invention relates to a compound of formula I, or a pharmaceutical acid addition salt thereof, for use in treating endometriosis and/or uterine leiomyoma. The present invention is further related to the use of a compound of formula I, or a pharmaceutical acid addition salt thereof, for the manufacture of a medicament for treating endometriosis and/or uterine leiomyoma.

The present invention further relates to a compound of formula II:

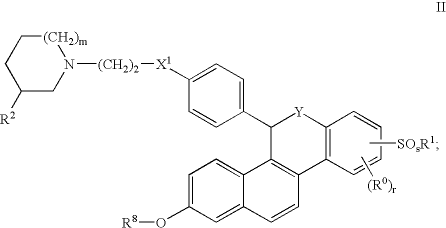

wherein:

m, r, R⁰, R¹, R², R³ and Y are as defined above for a formula I compound and

s is 0, 1 or 2;

R⁸ is H, C₁-C₆ alkyl, benzyl, SO₂CH₃, SO₂(n-C₄-C₆ alkyl) or COR³;

X¹ is O or NR⁹; and

R⁹ is H, C₁-C₆ alkyl or CO₂(C₁-C₆ alkyl); provided that if s is 2, then R⁸ is C₁-C₆ alkyl, SO₂CH₃ or benzyl or R⁹ is CO₂(C₁-C₆ alkyl); or an acid addition salt thereof; useful as an intermediate to a compound of formula I.

### DETAILED DESCRIPTION

Unless specified otherwise, reference hereafter to a “compound of formula I” includes the pharmaceutical acid addition salts thereof.

The compounds of the present invention have one or more chiral centers and may exist in a variety of stereoisomeric configurations. As a consequence of these chiral centers, the compounds of the present invention occur as racemates, mixtures of enantiomers and as individual enantiomers, as well as diastereomers and mixtures of diastereomers. All such racemates, enantiomers, and diastereomers are within the scope of the present invention.

For the purposes of the present invention, as disclosed and claimed herein, the following terms are defined below.

The term “halo” refers to fluoro, chloro, bromo and iodo. The term “C₁-C₆ alkyl” represents a straight, branched or cyclic hydrocarbon moiety having from one to six carbon atoms, e.g., methyl, ethyl, n-propyl, isopropyl, cyclopropyl, n-butyl, isobutyl, sec-butyl, t-butyl, cyclobutyl, pentyl, cyclopentyl, hexyl, cyclohexyl and the like. Moieties such as a cyclobutylmethylene are also included within the scope of a C₁-C₆ alkyl group. The term “C₁-C₄ alkyl” refers specifically to methyl, ethyl, n-propyl, isopropyl, cyclopropyl, cyclopropylmethyl, n-butyl, isobutyl, sec-butyl, t-butyl and cyclobutyl. The term “n-C₄-C₆ alkyl” refers specifically to n-butyl, n-pentyl and n-hexyl. A “C₁-C₆ alkoxy” group is a C₁-C₆ alkyl moiety connected through an oxy linkage.

The term “pharmaceutical” when used herein as an adjective means substantially non-deleterious.

A pharmaceutical “acid addition salt” is a salt formed by reaction of the free base form of a compound of formula I with a pharmaceutical acid, such as described in the Encyclopedia of Pharmaceutical Technology, editors James Swarbrick and James C. Boylan, Vol 13, 1996 “Preservation of Pharmaceutical Products to Salt Forms of Drugs and Absorption”. Specific salt forms include, but are not limited to the: acetate, benzoate, benzenesulfonate, 4-chlorobenzenesulfonate; citrate; ethanesulfonate; fumarate; d-gluconate; d-glucuronate; glutarate; glycolate; hippurate; hydrochloride; 2-hydroxyethanesulfonate; dl-lactate; maleate; d-malate; 1-malate; malonate; d-mandelate; 1-mandelate; methanesulfonate; 1,5 napthalenedisulfonate; 2-naphthalenesulfonate; phosphate; salicylate; succinate; sulfate; d-tartrate; 1-tartrate; and p-toluenesulfonate.

The term “patient” as used herein refers to female humans and non-human female animals such as companion animals (dogs, cats, horses and the like).

The terms “treating” and “treat” as used herein, means alleviating, ameliorating, preventing, prohibiting, restraining, slowing, stopping, or reversing the progression or severity of a pathological condition, or sequela thereof, described herein. The term “preventing” means reducing the likelihood that the recipient of a compound of formula I will incur, further incur or develop any of the pathological conditions, or sequela thereof, described herein.

The term “patient in need thereof” is a patient either suffering from the claimed pathological condition or sequela thereof, or is a patient at a recognized risk thereof, as determined by medical diagnosis, i.e., as determined by the attending physician.

As used herein, the term “effective amount” means an amount of a compound of formula I that is capable of treating the conditions described herein.

### Preferred Compounds and Embodiments of the Invention

Certain compounds of the invention are particularly interesting and are preferred. The following listing sets out several groups of preferred compounds. It will be understood that each of the listings may be combined with other listings to create additional groups of preferred compounds. The following numbering system will be used to describe the preferred positions of the SO₂R¹ moiety:

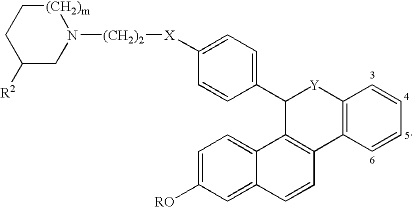

a) m is 1 or 2;

b) m is 1;

c) r is 0;

d) r is 1 and R⁰ is OH, CF₃, fluoro, C₁-C₄ alkyl or C₁-C₄ alkoxy;

e) r is 1 and R⁰ is fluoro;

f) R is H;

g) R is H or COR³ and R³ is C₁-C₆ alkyl, NHCH₃ or phenyl;

h) R is H or COR³ and R³ is C₁-C₄ alkyl, NHCH₃ or phenyl;

i) the —SO₂R¹ moiety is at the 4-position;

j) the —SO₂R¹ moiety is at the 5-position and R¹ is NR⁴R^{4a} or CF₃ and R⁴ is C₁-C₄ alkyl and R^{4a} is H or C₁-C₄ alkyl;

k) R¹ is C₁-C₄ alkyl, NR⁴R^{4a} or CF₃ and R⁴ is C₁-C₄ alkyl and R^{4a} is H or C₁-C₄ alkyl;

l) R¹ is methyl, ethyl, cyclopropyl, NHCH₃, N(CH₃)₂ or CF₃;

m) R¹ is methyl or N(CH₃)₂;

n) R¹ is methyl;

o) R¹ is N(CH₃)₂;

p) X is O;

q) X is NR⁷ and R⁷ is H or methyl;

r) Y is O;

s) the hydrochloride salt form.

With respect to the chiral center designated below:

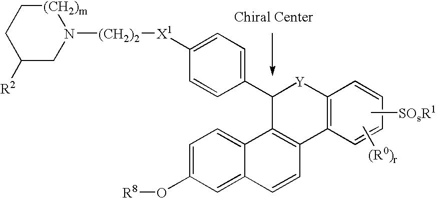

an enantiomeric excess (ee) of greater than 90% is preferred, an ee of greater than 95% is most preferred and an ee of greater than 99% is most especially preferred. Enantiomeric enrichment is readily determined by one of ordinary skill in the art using standard techniques and procedures, such as gas or high performance liquid chromatography with a chiral column (see, e.g., J. Jacques, et al., “Enantiomers, Racemates, and Resolutions”, John Wiley and Sons, Inc., 1981; E. L. Eliel and S. H. Wilen,” Stereochemistry of Organic Compounds”, (Wiley-Interscience 1994), and European Patent Application No. EP-A-838448, published Apr. 29, 1998). Of course, the preferred enantiomer is that which possesses favorable activity in the biological assays disclosed herein. Employing the chiral chromatography disclosed in the Examples below, the preferred enantiomer (the enantiomer with favorable activity) typically possesses the slower retention time, i.e., elutes second. In order to verify the identify of the preferred enantiomer in any given racemic mixture, the activity of the individual isomers should be verified in the biological assays described herein.

The preferred patient of treatment is a female human.

The compound of formula I is preferably formulated in a dosage unit form, i.e., in an individual delivery vehicle, for example, a tablet or capsule, prior to administration to the recipient woman.

The compound of formula I is preferably administered orally.

### Synthesis

The compound of formula I may be prepared as described in the following Examples and as described in Scheme 1 where R¹⁰ is fluoro or an alkyl protected hydroxy or thio moiety.

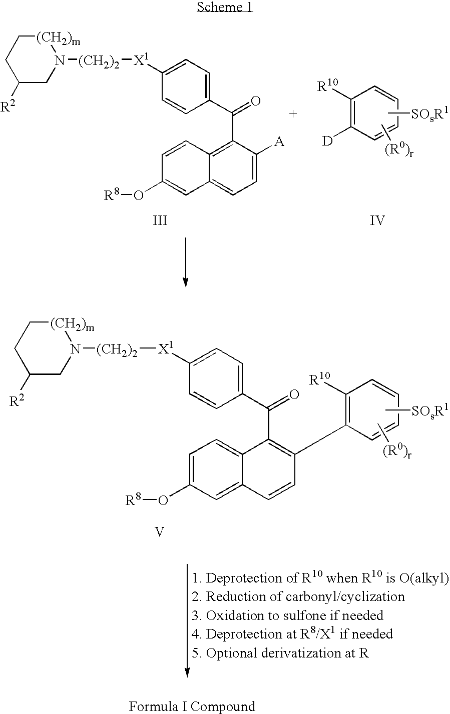

In Scheme 1, a compound of formula IV is reacted with a compound of formula III under usual “Suzuki” or “Stille” reaction conditions, i.e., wherein one of substituent “A” or “D” is a boronic acid/ester or alkyl stannane moiety and the other is a leaving group, e.g., chloro, bromo or iodo or a sulfonate group such as trifluoromethyl sulfonate to provide a compound of formula V.

When R¹⁰ is protected hydroxy, said hydroxy group is typically removed in order to promote the following reduction/cyclization reaction. Said protecting group may be removed via standard procedure, e.g., those described in the Examples below or as taught in the latest edition of Greene, Protective Groups in Organic Synthesis, John Wiley & Sons, New York, N.Y.). After removal of the hydroxy protecting group, the keto group found in the resulting product compound of formula V may then be reduced under standard conditions, e.g., employing borane to provide the corresponding compound of formula III where Z is CHOH. This reduced product may then be cyclized under standard conditions, e.g., when R¹⁰ is F, base catalyzation with potassium t-butoxide or when R¹⁰ is other than F, acid catalyzation with HCl, to provide the corresponding compound of formula I or II.

When s is 0 or 1, the cyclized product may be oxidized under standard conditions (see working examples below) to prepare the corresponding sulfone of formula I. When R⁸ is SO₂CH₃, C₁-C₆ alkyl or benzyl (preferably methyl, benzyl or SO₂CH₃) said hydroxy protecting groups may be removed under standard conditions (see, e.g., the procedures that follow or the latest edition of Greene, Protective Groups in Organic Synthesis, John Wiley & Sons, New York, N.Y.) to provide the compound of formula I where R is H. Similarly, when X¹ is NR⁹ and R⁹ is CO₂(C₁-C₆ alkyl), said amino protecting group may also be removed as taught in Greene. A formula I compound where R is H may be further derivatized employing standard acylation or sulfonylation methodology to prepare a compound of formula I where R is COR³ or SO₂(n-C₄-C₆ alkyl).

A compound of formula I may also be prepared by introducing an SR¹ moiety post-cyclization by nucleophilic displacement of fluorine followed by oxidation to the corresponding sulfone as described in Examples 8 and 9 below.

Compounds of formula III may be prepared as shown below or by procedures analogous to those found in the art. Compounds of formula IV are, in general, commercially available or can be prepared by procedures readily available to the ordinarily skilled synthetic organic chemist or as shown below.

### GENERAL EXPERIMENTAL DETAILS

Electrospray mass spectra may be obtained on a Finnigan LCQ Duo instrument using a mobile phase of 50% acetonitrile, 25% methanol, and 25% 2mM aqueous ammonium acetate. Preparative HPLC's may be obtained on a Gilson Preparative System with Unipoint Software and dual wavelength detection at 220 and 254 nm as well as Finnigan aQa MS. A 20-mm×250 -mm ODS-AQ column with a particle size of 15 microns may be used as the stationary phase. The eluent is a binary system of bottle A (0.1% trifluoroacetic acid (TFA), 1% isopropyl alcohol (IPA) in water) and bottle B (0.05% TFA, 1% IPA in acetonitrile). The standard method is a gradient of 30-95% B unless otherwise indicated. The compounds purified by this method are isolated as TFA salts.

Preparative HPLC's may also be obtained on a Biotage ParallelFlex system with proprietary dual wavelength detection and software. A 30-mm×150-mm or 19-mm×250 mm Xterra column with a particle size of 10 microns is used as the stationary phase and 10 mM NH₄⁺HCOO⁻/10 mM NH₄OH is used as mobile phase A and 100% acetonitrile is used as a mobile phase B.

### Preparation 1

### 1-Bromo-2-isopropoxy-4-methylthio-benzene

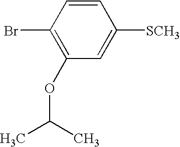

Add 2-Bromopropane (6.0 mL, 0.062 mol, Aldrich) and potassium carbonate (21.0 g, 0.156 mol) to a solution of 2-bromo-5-fluorophenol (10.0 g, 0.052 mol) in 80 mL of acetone. Heat the mixture in a 70° C. oil bath, and stir under reflux for 17 hours. Remove the solvents in vacuo, add 200 mL water, and extract the mixture 3 times with dichloromethane. Dry the combined organic layers over Na₂SO₄, and evaporate the solvents in vacuo.

Dissolve the residue (5.0 g, 21.45 mmol) in 25 mL of dimethylformamide (DMF) at room temperature. Add NaSCH₃ (1.87 g, 26.81 mmol) all at once. Fit the reaction vessel with a reflux condenser then heat to 60° C. with stirring for 2 hours. Cool the reaction to room temperature, then add 50 mL of H₂O, and extract the mixture 3 times with dichloromethane. Combine the organics, dry over Na₂SO₄ and remove the solvents in vacuo. Purify the residue by column chromatography on a 90 g SiO₂ cartridge, using 5% ethyl acetate (EtOAc)/hexanes at first, then 20% EtOAc/hexanes to give 3.99 g, (15.2 mmol, 71.2% yield) of the title compound.

### Preparation 2

### Trifluoromethanesulfonic acid 6-methoxy-1-[4-(2-piperidin-1-yl-ethoxy)-benzoyl]-naphthalen-2-yl ester

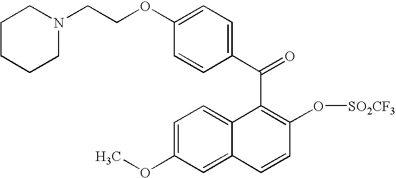

Dissolve 2,6-dimethoxynaphthalene (1.0 equivalent (eq)) in CH₂Cl₂ (5 volume equivalents) at ambient temperature in a dry round bottom flask equipped with stir bar, temperature probe and N₂ line. Cool the solution to 0° C. with an ice bath, and add 4-(2-piperidin-1-yl-ethoxy)-benzoyl chloride (1.1 eq). Add aluminum chloride (2.0 eq). Once the reaction is determined to be complete, quench the reaction slowly with 1 N NaOH and dilute with additional water and CH₂Cl₂. Wash the aqueous layer with CH₂Cl₂ (20 mL). Combine the organic extracts and wash with brine and dry (Na₂SO₄). Recrystallize the crude product from methanol to give (2,6-dimethoxy-naphthalen-1-yl)-[4-(2-piperidin-1-yl-ethoxy)-phenyl]-methanone.

Dissolve (2,6-dimethoxy-naphthalen-1-yl)-[4-(2-piperidin-1-yl-ethoxy)-phenyl]-methanone in CH₂Cl₂ (10 volume equivalents) in a 3-neck round bottom flask equipped with a pressure equalizing addition funnel, stir bar, and N₂ source. Cool the flask in an ice/brine bath and add 1.0 M BCl₃ solution in CH₂Cl₂ (1.2 eq) dropwise. After about 1 hour, quench the reaction with methanol (5 eq) and allow the reaction to warm to room temperature. Dilute the organic solution with CH₂Cl₂ (one volume eq) and add a 1.0 M NaHCO₃ solution (5 volume eq) and stir for one hour. Separate the aqueous and organic layers. Wash the aqueous layer with CH₂Cl₂ (one volume) and combine the organic layers. Wash with saturated NH₄Cl and dry over Na₂SO₄. Purify the product via column chromatography (50/1 silica gel) eluting with CH₂Cl₂/hexanes (3/1) to yield (2-hydroxy-6-methoxy-naphthalen-1-yl)-[4-(2-piperidin-1-yl-ethoxy)-phenyl]-methanone.

Dissolve (2-hydroxy-6-methoxy-naphthalen-1-yl)-[4-(2-piperidin-1-yl-ethoxy)-phenyl]-methanone in CH₂Cl₂ (10 volumes) in a three neck round bottom flask equipped with a stir bar and N₂ source and chill to 0° C. in an ice/brine bath. Add pyridine (1.3 equivalents) and trifluoromethanesulfonyl chloride (1.2 equivalents) via syringe over 15 minutes. After about 15 minutes, quench the reaction with H₂O (10 volumes), wash with 1 N aqueous HCl (5 volumes) and 1.0 N aqueous NaHCO₃, and dry over Na₂SO₄. Obtain the title compound in quantitative yield after concentration.

### Preparation 3

### [2-(2-Isopropoxy-4-methanesulfanyl-phenyl)-6-methoxy-naphthalen-1-yl]-[4-(2-piperidin-1-yl-ethoxy)-phenyl]-methanone

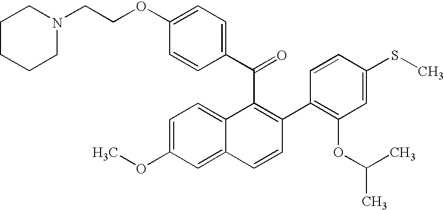

Dissolve trifluoro-methanesulfonic acid 6-methoxy-1-[4-(2-piperidin-1-yl-ethoxy)-benzoyl]-naphthalen-2-yl ester (1.9 g, 3.18 mmol) in 30 mL acetonitrile, and degas 3 times. Add bis-neopentylglycolato diborane (789 mg, 3.51 mmol), palladium acetate (107 mg, 0.48 mmol) and tricyclohexyl phosphine (200 mg, 0.717 mmol) all at once, and again degas the mixture. Stir for 5 minutes to completely dissolve the reagents. Add cesium fluoride (4.3 g, 28.6 mmol) all at once, and immediately plunge the reaction vessel into a 90° C. preheated oil bath. After 2-3 minutes, add 1-bromo-2-isopropoxy-4-methanesulfanyl-benzene (0.915 g, 3.51 mmol) in 5 mL acetonitrile. After stirring at 90° C. for 20 minutes, cool the reaction, filter through a 2 g SiO₂ plug, and remove the solvents in vacuo. Dilute the residue with dichloromethane (DCM)/isopropanol (i-PrOH) (4:1) (100 mL), wash once with 100 mL H₂O, once with 100 mL brine, dry over Na₂SO₄, and remove the solvents in vacuo. Isolate the title compound via silica gel chromatography using 20% tetrahydrofuran (THF)/hexanes with 1% (2N NH₃/methanol (MeOH)) initially, then 20% THF/hexanes with 2% (2N NH₃/MeOH) to elute the compound to give 960 mg (52.8% yield). MS: 570 (M+1).

### EXAMPLE 1

### 8-Methylsulfanyl-5-[4-(2-piperidin-1-yl-ethoxy)-phenyl-5H-6-oxa-chrysen-2-ol hydrochloride

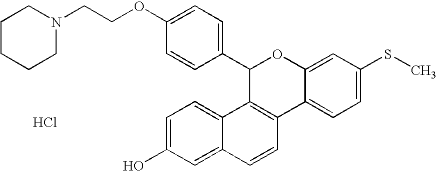

Dissolve [2-(2-isopropoxy-4-methanesulfanyl-phenyl)-6-methoxy-naphthalen-1-yl]-[4-(2-piperidin-1-yl-ethoxy)-phenyl]-methanone (960 mg, 1.68 mmol) in dichloromethane. Bubble HCl (g) through the solution for 5 minutes. Remove solvents in vacuo, dissolve the residue in dichloromethane (20 mL), cool to −10° C., and add 0.637 mL (6.74 mmol) of BBr₃ dropwise. Stir the reaction for 0.5 hours at −10° C., then pour into a solution of 100 mL of cold aqueous NaHCO₃. Extract the mixture 3 times with 20 mL of dichloromethane (DCM), 3 times with 20 mL DCM/i-PrOH (4:1), and then remove the solvents in vacuo.

Dissolve the residue in THF, place under N₂, and add lithium aluminum hydride (1.0 M, 6.74 mL, 6.74 mmol) dropwise, slowly. Stir for one hour at room temperature. Add H₂O slowly to quench. Add 30 mL 1.0N HCl, and stir at room temperature for 1 hour. Isolate via silica gel chromatography (20% THF/hexanes with 0% to 10% (2N NH₃/MeOH)). Isolate the title compound to give 355 mg, 0.71 mmol, 42.4% yield.

### Preparation 4

### 1-Bromo-2-isopropoxy-4-methanesulfonyl-benzene

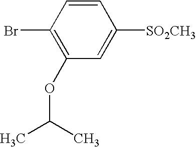

Dissolve the compound of preparation 1 (3.99 g, 15.28 mmol) in acetic acid (HOAc) at room temperature, under N₂. Add NaBO₃.H₂O (3.35 g, 33.61 mmol) all at once, and stir the reaction at room temperature for 17 hours. Remove the solvent in vacuo and add H₂O (30 mL). Extract the mixture 3 times with dichloromethane, combine the organics, dry over Na₂SO₄, and remove the solvents in vacuo to give the title product (4.06 g, 12.21 mmol, 79.9% yield.

### Preparation 5

### [2-(2-Isopropoxy-4-methanesulfonyl-phenyl)-6-methoxy-naphthalen-1-yl]-[4-(2-piperidin-1-yl-ethoxy)-phenyl]-methanone

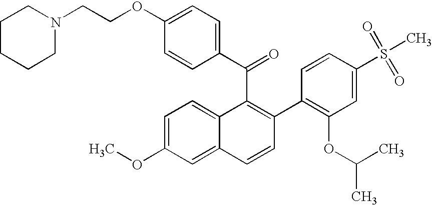

Dissolve trifluoromethanesulfonic acid 6-methoxy-1-[4-(2-piperidin-1-yl-ethoxy)-benzoyl]-naphthalen-2-yl ester (6.0 g, 11.16 mmol) in 100 mL acetonitrile, and degas 3 times. Add bis-neopentylglycolato diborane (2.76 g, 12.3 mmol), palladium acetate (375 mg, 1.7 mmol) and tricyclohexyl phosphine (703 mg, 2.5 mmol) all at once, and again, degas the mixture. Stir for 5 minutes to completely dissolve the reagents. Add cesium fluoride (15.17 g, 100 mmol), all at once, and immediately plunge the reaction vessel into a 90° C. preheated oil bath. After 2-3 minutes, add 1-bromo-2-isopropoxy-4-methanesulfonyl-benzene (3.6 g, 12.3 mmol) dissolved in 20 mL acetonitrile. After stirring at 90° C. for 20 minutes, cool the reaction, filter through a 2 g SiO₂ plug, and remove the solvents in vacuo. Dilute the residue with DCM/i-PrOH (4:1) (500 mL), wash once with 100 mL H₂O, once with 100 mL brine, dry over Na₂SO₄, and remove the solvents in vacuo to give the title compound (7.1 g, 99% yield). MS: 602 (M+1).

### Preparation 6

### [6-Hydroxy-2-(2-hydroxy-4-methanesulfonyl-phenyl)-naphthalen-1-yl]-[4-(2-piperidin-1-yl-ethoxy)-phenyl]-methanone

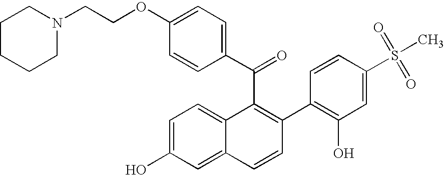

Dissolve [2-(2-isopropoxy-4-methanesulfonyl-phenyl)-6-methoxy-naphthalen-1-yl]-[4-(2-piperidin-1-yl-ethoxy)-phenyl]-methanone (7.1 g, 11.16 mmol) in acetonitrile, and add 2.79 mL of 5N HCl. Stir the reaction for 30 minutes at room temperature and remove the solvents in vacuo. Dissolve the residue in dichloromethane (1L), cool to −10° C., and add 5.28 mL (55.8 mmol) of BBr₃ dropwise. Stir the reaction for 2 hours at −10° C., at which time add another 3.17 mL (33.48 mmol) of BBr₃. Stir for an additional 3 hours, then pour into a solution of 500 mL of cold aqueous NaHCO₃. Extract the mixture 3 times with 100 mL of DCM, 3 times with 100 mL DCM/i-PrOH (4:1), and then filter the aqueous layer through a glass frit. Dry the filtrate under vacuum for 17 hours to give the title product (3.18 g, 5.83 mmol, 52.2% yield). MS: 546 (M+1).

### EXAMPLE 2

### 8-Methanesulfonyl-5-[4-(2-piperidin-1-yl-ethoxy)-phenyl]-5H-6-oxa-chrysen-2-ol

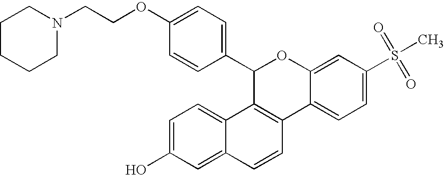

Stir [6-hydroxy-2-(2-hydroxy-4-methanesulfonyl-phenyl)-naphthalen-1-yl]-[4-(2-piperidin-1-yl-ethoxy)-phenyl]-methanone (3.18 g, 5.83 mmol) to dissolve in THF at room temperature, under N₂. Add lithium aluminum hydride (1.0 M, 17.5 mL, 17.4 mmol) dropwise, slowly and stir the reaction at room temperature for 2 hours. Carefully add HCl (1.0 N, 29.14 mL, 29.1 mmol) to the reaction mixture. After the addition is complete, add 2.0 mL of 5N HCl and stir the reaction at room temperature for 2 hours. Heat the mixture to 50° C. on a water bath, and stir for 20 minutes. Evaporate the THF in vacuo, and add saturated aqueous NaHCO₃ solution until the mixture is neutral. Extract 3 times with 100 mL DCM/i-PrOH (4:1). Combine the organic layers over Na₂SO₄, and remove the solvents in vacuo.

Purify through a 120 g SiO₂ cartridge using 20% THF/Hexanes with 5% (2N NH₃/MeOH) with a manual gradient to 40% THF/Hexanes with 12% (2N ammonia in methanol NH₃/MeOH) to isolate the title compound (3.8 g, 2.2 mmol, 38.8% yield). LCMS: 531 (M+1), 529 (M−1).

### EXAMPLE 3

### 8-Methanesulfonyl-5-[4-(2-piperidin-1-yl-ethoxy)-phenyl]-5H-6-oxa-chrysen-2-ol, mono-Hydrochloride salt

Stir to dissolve 8-methanesulfonyl-5-[4-(2-piperidin-1-yl-ethoxy)-phenyl]-5H-6-oxa-chrysen-2-ol in dichloromethane, bubble HCl gas through for 3 minutes, and stir the solution for 10 minutes. Add ethyl acetate and remove the solvents in vacuo to give the title compound. LCMS: 100%, 530 (M+1), 528 (M−1). Separate the racemic mixture into its constituent enantiomers by chiral chromatography (Examples 3a and 3b).

### Preparation 7

### 1-Bromo-2-isopropoxy-4-fluoro-benzene

Add 2-bromopropane (3.14 mL, 33 mmol) and potassium carbonate (9.20 g, 67 mmol) to a solution of 2-bromo-5-fluorophenol (5.80 g, 30 mmol) in anhydrous acetone (60 mL). Heat the mixture at reflux for 24 hours. Filter the reaction mixture and concentrate the filtrate in vacuo. Purify by silica gel chromatography, eluting with hexane, to give the title product (6.56 g, 92% yield).

### Preparation 8

### 1-Bromo-2-isopropoxy-4-benzylthiobenzene

Cool a suspension of sodium hydride (1.63 g, 68 mmol) in dry N,N-dimethyl formamide (25 mL) in an ice water bath, and then add benzyl mercaptan (4.44 g, 34 mmol) slowly. After 30 minutes add an additional 5 mL of N,N-dimethyl formamide followed by 1-bromo-2-isopropoxy-4-fluoro-benzene (7.93 g, 34 mmol). Warm the reaction mixture to room temperature and stir for 24 hours. Dilute the mixture with DCM (100 mL), wash with H₂O, NaHCO₃, dried over Na₂SO₄, and evaporate the solvent in vacuo. Purify by silica gel chromatography eluting with hexane to yield the title product (0.994 g, 9% yield).

### Preparation 9

### 1-Bromo-2-isopropoxy-4-N,N-dimethyl sulfonamide

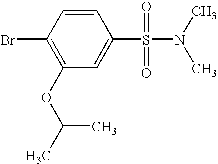

Cool a solution of 1-bromo-2-isopropoxy-benzylthiobenzene (990 mg, 3.07 mmol) in a mixture of dichloromethane/acetic acid/water (12.5%/5%/2.5%, 30 mL, 0.1M) and maintain at 5° C. Slowly bubble gaseous chlorine into the solution for 3 hours. Dilute the solution with DCM (400 mL) and wash twice with H₂O, once with saturated aqueous NaHCO₃, and again with H₂O. Dry the DCM phase over Na₂SO₄ and concentrate in vacuo to afford the 1-bromo-2-isopropoxy-4-sulfonylchloride intermediate. Dissolve this intermediate (0.96 g, 3.0 mmol) in THF with 2M dimethylamine in THF (7.5 ml, 15 mmol) and heat at 60° C. under a N₂ atmosphere for 30 minutes. Dilute the solution with ether, wash twice with 0.1 N HCl, saturated aqueous NaHCO₃ and H₂O. Dry the ether phase over Na₂SO₄ and concentrate in vacuo. Purify by silica gel chromatography eluting with CH₂Cl₂ to give the title compound (560 mg, 1.74 mmol, 57% yield).

### Preparation 10

### [2-(2-isopropoxy-4-,N,N-dimethyl sulfonamide-phenyl)-6-methoxy-1-napthalen-1-yl]-[4-(2-piperidin-1-yl-ethoxy)-phenyl]-methanone

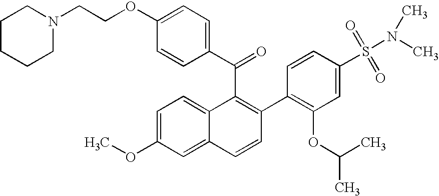

Dissolve 2-trifluoromethanesulfonic acid 6-methoxy-1-[4-(2-piperidin-1-yl-ethoxy)-benzoyl]-napthalen-2-yl ester (0.61 g, 1.13 mmol) in 15 mL acetonitrile and degas under N₂. Add bis-neopentylglycolato diborane (0.28 g, 1.24 mmol), palladium acetate (0.04 g, 0.186 mmol), and tricyclohexyl phosphine (0.087 g, 0.28 mmol), and degas again under N₂. Stir the mixture until all reagents dissolve. Add cesium fluoride (1.71 g, 11.3 mmol), and immediately place the reaction into a 90° C. preheated oil bath. After 3-7 minutes, add 1-bromo-2-isopropoxy-4-N,N-dimethyl sulfonamide ( 0.4 g, 1.24 mmol) in 10 mL acetonitrile and the stir the mixture at 90° C. for 6 hours. Cool the mixture, filter through a thin layer silica gel and wash the silica with acetonitrile. Concentrate the filtrate in vacuo and purify by silica gel chromatography (20% THF/Hexane with gradient 0 to 5% 2N NH₃/MeOH) to yield the title compound (363 mg, 51% yield). MS: 631 (M+1).

### Preparation 11

### [2-(2-hydroxy-4-,N,N-dimethyl sulfonamide-phenyl)-6-hydroxy-1-napthalen-1-yl]-[4-(2-piperidin-1-yl-ethoxy)-phenyl]-methanone

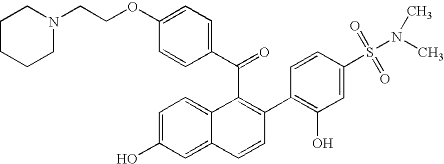

Slowly bubble gaseous HCl into a solution of [2-(2-isopropoxy-4-,N,N-dimethyl sulfonamide-phenyl)-6-methoxy-1-napthalen-1-yl]-[4-(2-piperidin-1-yl-ethoxy)-phenyl]-methanone (0.363 g, 0.58 mmol) in DCM (10 mL). Stir the solution for 15 minutes, and concentrate in vacuo. Dissolve the residue in DCM (10 mL), cool to 0° C., and add boron tribromide (0.22 ml, 2.3 mmol) dropwise. Stir the reaction at 0° C. for 2 hours, and then stir at room temperature for 1 hour. Add saturated aqueous NaHCO₃ (100 mL), extract the aqueous mixture 3 times with DCM/i-PrOH (4:1), dry over Na₂SO₄, and concentrate in vacuo to give the title product (650 mg). MS: 575 (M+1).

### EXAMPLE 4

### 8-N,N-dimethyl sulfonamide-5-[4-(2-piperidin-1-yl-ethoxy)-phenyl]-5H-6-oxachrysen-2-ol trifluoroacetate salt

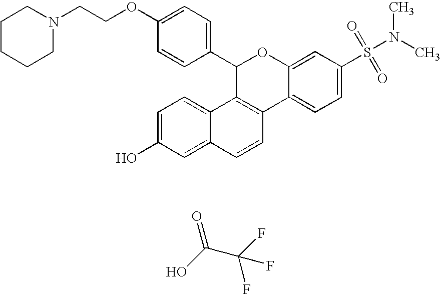

Dissolve [2-(2-hydroxy-4-,N,N-dimethyl sulfonamide-phenyl)-6-hydroxy-1-napthalen-1-yl]-[4-(2-piperidin-1-yl-ethoxy)-phenyl]-methanone (650 mg, 1.13 mmol) in THF (15 mL) at room temperature, under N₂. Slowly add lithium aluminum hydride dropwise and stir the reaction at room temperature for 24 hours. Quench the mixture with H₂O (a few drops) until no further gas evolution, and then add HCl (5N, 6 mL). After aging the mixture at room temperature for four hours, remove the THF in vacuo, and add saturated aqueous NaHCO₃ until the mixture is neutral to hydrion paper. Extract the aqueous phase 3 times with DCM/i-PrOH (4:1) and dry the combined organic layers over Na₂SO₄. Evaporation of solvent yields a crude solid which is purified by silica gel chromatography eluting with 5% 2N NH₃/MeOH in CH₂Cl₂ to give the title product (140 mg, 0.25 mmol, 22% yield). MS:559.2 (M+1).

### Preparation 12

### 1-bromo-2-isopropoxy-4-N-methyl sulfonamide

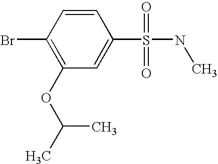

Dissolve 1-bromo-2-isopropoxy-4-sulfonylchloride (0.73 g, 2.3 mmol) in 2M methylamine in THF (17.25 mL, 34.5 mmol) and heat at 60° C. under a N₂ atmosphere for 30 minutes. Dilute the solution with DCM, wash twice with 0.1 N HCl, saturated aqueous NaHCO₃, and H₂O. Dry the ether phase over Na₂SO₄ and concentrate in vacuo. Purify by silica gel chromatography eluting with CH₂Cl₂ to yield the title compound (390 mg, 1.27 mmol, 54% yield). MS: 307 (M−1).

### Preparation 13

### [2-(2-isopropoxy-4-N-methyl sulfonamide-phenyl)-6-methoxy-1-napthalen-1-yl]-[4-(2-piperidin-1-yl-ethoxy)-phenyl]-methanone

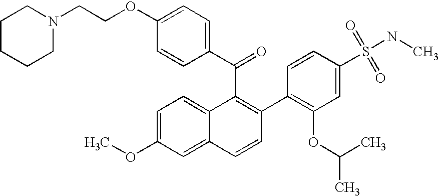

Dissolve 2-trifluoromethanesulfonic acid 6-methoxy-1-[4-(2-piperidin-1-yl-ethoxy)-benzoyl]-napthalen-2-yl ester (590 mg, 1.10 mmol) in 15 mL of acetonitrile, and degas under N₂. Add bis-neopentylglycolato diborane (270 mg, 1.21 mmol), palladium acetate (0.038 g, 0.16 mmol), and tricyclohexyl phosphine (69 mg, 0.25 mmol) and, again, degas under N₂. Stir the mixture until the reagents are completely dissolved. Add cesium fluoride (1.5 g, 9.9 mmol) and place the reaction immediately into a 90° C. preheated oil bath. After 3-7 minutes, add 1-bromo-2-isopropoxy-4-N-methyl sulfonamide (370 mg, 1.21 mmol) in 10 mL acetonitrile. After stirring at 90° C. for 24 hours, cool the reaction, filter through a thin layer of silica gel with acetonitrile and concentrate the filtrate in vacuo. Purify the dark solid by silica gel chromatography eluting with 20% THF/hexanes with gradient from 5 to 10% 2N NH₃/MeOH to yield the title product (268 mg, 40% yield). MS: 617.26 (M+1).

### Preparation 14

### [2-(2-hydroxy-4-N-methyl sulfonamide-phenyl)-6-hydroxy-1-napthalen-1-yl]-[4-(2-piperidin-1-yl-ethoxy)-phenyl]-methanone

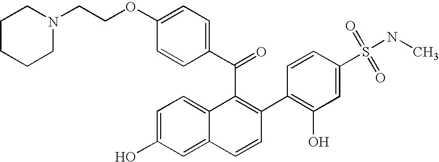

Slowly bubble gaseous HCl into a solution of [2-(2-isopropoxy-4-,N-methyl sulfonamide-phenyl)-6-methoxy-1-napthalen-1-yl]-[4-(2-piperidin-1-yl-ethoxy)-phenyl]-methanone (0.267 g, 0.43 mmol) in DCM (10 mL). Stir the solution for 15 minutes and concentrate in vacuo. Dissolve the residue in DCM (10 mL), cool to 0° C. and add boron tribromide (0.164 ml, 1.73 mmol) dropwise. Stir the reaction at 0° C. for 2 hours and then stir at room temperature for 1 hour. Add saturated aqueous NaHCO₃ (100 mL), extract the aqueous mixture 3 times with CH₂Cl₂/i-PrOH (4:1), dry over Na₂SO₄, and evaporate in vacuo to give the title compound (160 mg, 67% yield).

### EXAMPLE 5

### 8-N-methyl sulfonamide-5-[4-(2-piperidin-1-yl-ethoxy)-phenyl]-5H-6-oxacbrysen-2-ol

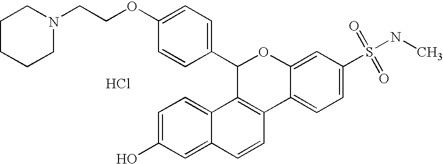

Dissolve [2-(2-hydroxy-4-,N-methyl sulfonamide-phenyl)-6-hydroxy-1-napthalen-1-yl]-[4-(2-piperidin-1-yl-ethoxy)-phenyl]-methanone (160 mg, 0.29 mmol) in room temperature THF (10 mL), under N₂. Slowly add lithium aluminum (1.43 mL, 1.43 mmol) hydride dropwise and stir the mixture at room temperature for 24 hours. Quench the mixture with H₂O (few drops) until no further gas evolution and add HCl (5N, 1.4 mL). Stir the reaction at 50° C. in a water bath for one hour. Remove the THF in vacuo and add saturated aqueous NaHCO₃ until the mixture is neutral to hydrion paper. Extract the aqueous phase 3 times with DCM/i-PrOH (4:1) and dry the combined organic phase over Na₂SO₄. Evaporate the solvent and purify by silica gel chromatography using a gradient of 0 to 5% 2N NH₃/MeOH in CH₂Cl₂ to give the title compound (95 mg, 60% yield). MS: 545 (M+1).

### Preparation 15

### 1-{2-[4-(8-fluoro-2-methoxy-5H-6-oxa-chrysen-5-yl)-phenoxy]-ethyl}-piperidine

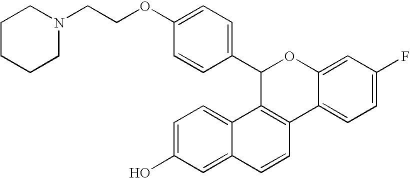

Dissolve trifluoromethanesulfonic acid 6-methoxy-1-[4-(2-piperidin-1-yl-ethoxy)-benzoyl]-naphthalen-2-yl ester (1.0 g, 1.86 mmol) in 25 mL acetonitrile and degas 3 times. Add bis-neopentylglycolato diborane (525 mg, 2.33 mmol), palladiumn acetate (63 mg, 0.28 mmol) and tricyclohexyl phosphine (117 mg, 0.42 mmol) all at once, and, again, degas the mixture. Stir for 5 minutes to completely dissolve the reagents. Add cesium fluoride (2.54 g, 16.7 mmol), all at once, and immediately plunge the reaction vessel into a 90° C. preheated oil bath. After 2-3 minutes, add 1-bromo-2-benzyloxy-4-fluorobenzene (654 mg, 2.33 mmol) in 5 mL acetonitrile. After stirring at 90° C. for 18 hours, cool the reaction, filter through a 2 g silica gel plug, and remove the solvents in vacuo. Dilute the residue with DCM/isopropanol (4:1) (40 mL), wash once with H₂O, once with brine and dry the organic layer over Na₂SO₄. After evaporation of solvent, isolate [2-(2-benzyloxy-4-fluoro-phenyl)-6-methoxy-naphthalen-1-yl]-[4-(2-piperidin-1-yl-ethoxy)-phenyl]-methanone via silica gel chromatography using DCM with 2% (2N NH₃/MeOH) to give 495 mg, 45% yield. Electrospray MS: 590 (M+1).

Dissolve [2-(2-benzyloxy-4-fluoro-phenyl)-6-methoxy-naphthalen-1-yl]-[4-(2-piperidin-1-yl-ethoxy)-phenyl]-methanone (470 mg, 0.80 mmol) in 20 ml dry THF under a nitrogen atmosphere. Add lithium aluminum hydride (1.0 M in THF, 1.2 mL, 1.2 mmol) dropwise, slowly. Stir for 1.5 hours at room temperature. To quench the reaction, add 0.5 ml H₂O dropwise, followed by 0.5 ml 1 N NaOH solution, then 1.5 ml H₂O. The mixture is then partitioned between ethyl acetate and water. Extract the aqueous layer two more times with 25 mL ethyl acetate, wash the combined organic layers with brine and dry over anhydrous sodium sulfate. Evaporation of the solvent yields [2-(2-Benzyloxy-4-fluoro-phenyl)-6-methoxy-naphthalen-1-yl]-[4-(2-piperidin-1-yl-ethoxy)-phenyl]-methanol, 0.47 g, 99% yield. Electrospray MS: 592 (M+1).

Dissolve [2-(2-Benzyloxy-4-fluoro-phenyl)-6-methoxy-naphthalen-1-yl]-[4-(2-piperidin-1-yl-ethoxy)-phenyl]-methanol (265 mg, 0.45 mmol) in 25 ml absolute ethanol under a nitrogen atmosphere. Add 60 mg of 10% palladium on carbon, wet (Degussa type E101 NE/W) and stir at room temperature under a blanket of hydrogen for six hours. Filter the mixture through a diatomaceous earth pad and evaporate the filtrate to give a crude foam residue.

Dissolve this residue in 8 ml of THF and 2.5 ml of 1 N aqueous HCl and stir at room temperature for 24 hours. Neutralize the mixture with saturated aqueous Na₂CO₃ and partition between ethyl acetate and water. Extract the aqueous layer twice more with 25 ml ethyl acetate and wash the combined organic layers with brine and dry over anhydrous sodium sulfate. The residue obtained after evaporation of the solvent is purified by radial chromatography (silica, CH₂Cl₂, gradient from 2% to 5% 2M NH₃ in methanol) to yield the title compound, 110 mg, 53%. Electrospray MS: 484 (M+1).

### EXAMPLE 6

### 8-Ethanesulfanyl-5-[4-(2-piperidine-1-yl-ethoxy)-phenyl]-5H-6-oxa-chrysen-2-ol

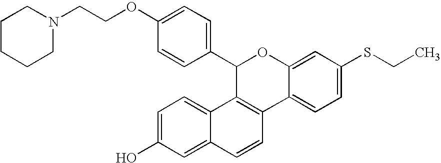

Dissolve 1-{2-[4-(8-fluoro-2-methoxy-5H-6-oxa-chrysen-5-yl)-phenoxy]-ethyl}-piperidine (172 mg, 0.356 mmol) in 5 ml dry dimethylformamide with sodium ethanethiolate (300 mg, 3.56 mmol) under a nitrogen atmosphere and heat to 90° C. for 17 hours. Reduce the volume by 75% by distillation of solvent and partition the remaining residue between water and 15% isopropanol in chloroform. Wash the aqueous layer twice more with 15% isopropanol in chloroform, wash the combined organic layers with brine and dry over anhydrous sodium sulfate. Purify the residue obtained after evaporation of solvent by radial chromatography (silica, DCM/2M ammonia in methanol, gradient from 98:2 to 94:6 to give 164 mg (88%) of the title product. Electrospray M. S. m+1=512.

### EXAMPLE 7

### 8-Ethanesulfonyl-5-[4-(2-piperidin-1-yl-ethoxy)-phenyl]-5H-6-oxa-chrysen-2-ol hydrochloride salt

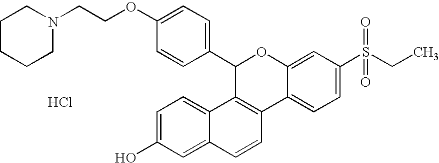

Dissolve 8-ethanesulfanyl-5-[4-(3-piperidin-1-yl-ethoxy)-phenyl]-5H-6-oxa-chrysen-2-ol (160 mg, 0.313 mmol) in 4 ml glacial acetic acid, add sodium perborate (66 mg, 0.657 mmol) and stir at room temperature for 16 hours. Evaporate the solvent and purify the residue by radial chromatography (silica, DCM/2M ammonia in methanol, gradient from 98:2 to 95:5). Dissolve the purified residue obtained after evaporation of solvent in DCM and a small amount of HCl gas bubbled into the solution. Dilute the mixture with ethyl acetate and evaporate the solvent to give the title compound (75 mg, 41%). Electrospray M.S. m+1=544.

### Preparation 16

### 3-Bromo-4-methoxy-N,N-dimethylbenzenesulfonamide

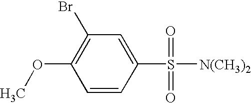

Combine 3-bromo-4-methoxybenzenesulfonyl chloride (1.0 g, 3.5 mmol) and dimethylamine (8.75 ml of 1M solution in THF, 8.75 mmol) in 25 ml anhydrous THF and heat under reflux in a nitrogen atmosphere for 30 minutes. Concentrate the cooled mixture in vacuo and partition the residue between ethyl acetate and water. Wash the organic layer with 0.1 N HCl solution, 0.1 N NaOH solution and brine and dry over

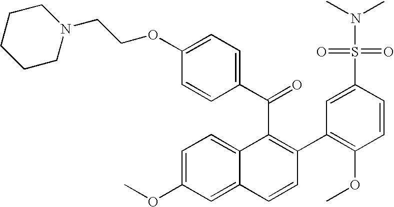

anhydrous sodium sulfate. Evaporate the solvent and recrystallize the crude solid from ethyl acetate/hexane to yield the title compound (680 mg, 66% yield).

### Preparation 17

### 4-Methoxy-3-{6-methoxy-1-[4-(2-piperidin-1-yl-ethoxy)-benzoyl]-naphthalen-2-yl}-N,N-dimethyl-benzenesulfonamide

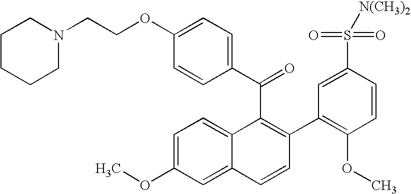

Dissolve 2-trifluoromethanesulfonic acid 6-methoxy-1-[4-(2-piperidin-1-yl-ethoxy)-benzoyl]-napthalen-2-yl ester (590 mg, 1.10 mmol) in 15 mL acetonitrile and degas under N₂. Add bis-neopentylglycolato diborane (0.31 g, 1.38 mmol), palladium acetate (0.037 g, 0.165 mmol), and tricyclohexyl phosphine (0.069 g, 0.25 mmol), and degas again under N₂. Stir the mixture until all reagents dissolve. Add cesium fluoride (1.50 g, 9.9 mmol), and immediately place the reaction into a 90° C. preheated oil bath. After 3-7 minutes, add 3-bromo-4-methoxy-N,N-dimethylbenzenesulfonamide (360 mg, 1.21 mmol) in 10 mL acetonitrile and stir the mixture at 90° C. for 6 hours. Cool the mixture, filter through a thin layer silica gel and the silica washed with acetonitrile. Concentrate the filtrate in vacuo to give a quantitative crude yield of the title product, electro spray MS: 603 (M+1).

### EXAMPLE 8

### 2-Hydroxy-5-[4-(2-piperidin-1-yl-ethoxy)-phenyl]-5H-6-oxa-chrysen-9-sulphonic acid dimethylamide hydrochloride salt

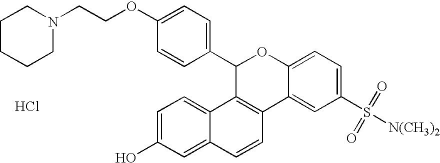

Slowly bubble gaseous HCl into a solution of 4-methoxy-3-{6-methoxy-1-[4-(2-piperidin-1-yl-ethoxy)-benzoyl]-naphthalen-2-yl}-N,N-dimethyl-benzenesulfonamide (710 mg crude, 1.1 mmol) in DCM (15 mL). Stir the solution for 15 minutes and concentrate in vacuo. Dissolve the residue in DCM (15 mL), cool to 0° C. and add boron tribromide (2.48 g, 9.9 mmol) dropwise. Stir the reaction at 0° C. for 1.5 hours and then stir at room temperature for 20 minutes. Add saturated aqueous NaHCO₃ (100 ML), extract the aqueous mixture 3 times with CHCl₃/i-PrOH (4:1), dry the combined organic layers over Na₂SO₄ and concentrate in vacuo. Purify the residue by radial chromatography (silica, CH₂Cl₂/2M NH₃ in methanol, gradient from 2% to 5%) to give 4-hydroxy-3-{6-hydroxy-1-[4-(2-piperidin-1-yl-ethoxy)-benzoyl]-naphthalen-2-yl}-N,N-dimethyl-benzenesulfonamide (0.36 g, 57%). Electrospray MS: 575 (M+1).

Dissolve 4-hydroxy-3-{6-hydroxy-1-[4-(2-piperidin-1-yl-ethoxy)-benzoyl]-naphthalen-2-yl}-N,N-dimethyl-benzenesulfonamide (360 mg, 0.63 mmol) at room temperature in THF (10 mL), under N₂. Slowly add lithium aluminum hydride (1M solution in THF, 1.25 ml, 1.25 mmol) dropwise and stir the reaction at room temperature for 2.5 hours. Quench the mixture with H₂O (a few drops) until no further gas evolution and add HCl (1N, 3 mL). After stirring the mixture at room temperature for two hours, remove the THF in vacuo, and add saturated aqueous NaHCO₃ until the mixture is neutral to hydrion paper. Extract the aqueous phase is 3 times with CH₂Cl₂/i-PrOH (4:1) and dry the combined organic layers over Na₂SO₄. Evaporate the solvent to yield a crude solid and purify by radial chromatography on silica eluting with a gradient of 2% to 5% 2N NH₃/MeOH in CH₂Cl₂. After evaporation of the solvent, which yields the free base, redissolve the residue in a small amount of acetonitrile and add five drops 1N HCl. Evaporation of solvent yields the title product (170 mg, 46% yield), electrospray MS: 559 (M+1, parent ion).

### Preparation 18

### 3-Bromo-4-methoxy-N-methylbenzenesulfonamide

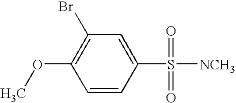

Combine 3-bromo-4-methoxybenzenesulfonyl chloride (1.0 g, 3.5 mmol) and methylamine (4.4 ml of 2M solution in THF, 8.8 mmol) in 25 ml anhydrous THF and heat under reflux in a nitrogen atmosphere for 30 minutes. Concentrate the cooled mixture in vacuo and partition the residue between ethyl acetate and water. Wash the organic layer with 0.1 N HCl solution, 0.1 N NaOH solution and brine and dry over anhydrous sodium sulfate. Evaporate the solvent and recrystallize the crude solid from ethyl acetate/hexane to yield the title compound (340 mg, 35% yield).

### Preparation 19

### 4-Methyoxy-3-{6-methoxy-1-[4-(2-piperidin-1-yl-ethoxy)-benzoyl]-naphthalen-2-yl}-N-methyl-benzenesulfonamide

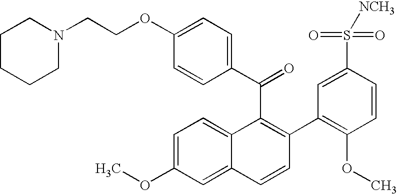

Dissolve 2-trifluoromethanesulfonic acid 6-methoxy-1-[4-(2-piperidin-1-yl-ethoxy)-benzoyl]-napthalen-2-yl ester (590 mg, 1.10 mmol) in 15 mL acetonitrile and degas under N₂. Add bis-neopentylglycolato diborane (0.31 g, 1.38 mmol), palladium acetate (0.037 g, 0.165 mmol), and tricyclohexyl phosphine (0.069 g, 0.25 mmol), and degas again under N₂. Stir the mixture until all reagents dissolve. Add cesium fluoride (1.50 g, 9.9 mmol), and immediately place the reaction into a 90° C. preheated oil bath. After 3-7 minutes, add 3-bromo-4-methoxy-N-methylbenzenesulfonamide (340 mg, 1.21 mmol) in 10 mL acetonitrile and stir the mixture 90° C. for 48 hours. Cool the mixture, filter through a thin layer of silica gel and wash the silica with acetonitrile. Concentrate the filtrate in vacuo and purify the residue by radial chromatography (silica, CH₂Cl₂/2M NH₃ in methanol, gradient from 2% to 6%) to yield the title product, 260 mg, 41% yield. Electrospray MS: 589 (M+1).

### EXAMPLE 9

### 2-Hydroxy-5-[4-(2-piperidin-1-yl-ethoxy)-phenyl]-5H-6-oxa-chrysen-9-sulfonic acid methylamide hydrochloride salt

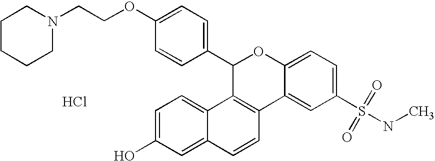

Slowly bubble gaseous HCl into a solution of 4-methoxy-3-{6-methoxy-1-[4-(2-piperidin-1-yl-ethoxy)-benzoyl]-naphthalen-2-yl}-methyl-benzenesulfonamide (260 mg, 0.44 mmol) in DCM (5 mL). Stir the solution for 15 minutes and concentrate in vacuo. Dissolve the residue again in DCM (5 mL), cool to 0° C. and add boron tribromide (880 mg, 3.5 mmol) dropwise. Stir the reaction at 0° C. for 2 hours then stir at room temperature for 1 hour. Add saturated aqueous NaHCO₃ (100 mL) and extract the aqueous mixture 3 times with CH₂Cl₂/i-PrOH (4:1). Dry the combined organic layers over Na₂SO₄ and evaporate in vacuo to give 4-hydroxy-3-{6-hydroxy-1-[4-(2-piperidin-1-yl-ethoxy)-benzoyl]-naphthalen-2-yl}-N-methyl-benzenesulfonamide (250 mg, quantitative crude yield). electrospray MS: 561 (M+1).

Dissolve 4-hydroxy-3-{6-hydroxy-1-[4-(2-piperidin-1-yl-ethoxy)-benzoyl]-naphthalen-2-yl}-N-methyl-benzenesulfonamide (250 mg, 0.44 mmol) at room temperature in THF (5 mL), under N₂. Slowly add lithium aluminum hydride (1M solution in THF, 0.88 ml, 0.88 mmol) dropwise and stir the reaction at room temperature for 2.5 hours. Quench the mixture with H₂O (a few drops) until no further gas evolution, and add HCl (1N, 3 mL). After stirring the mixture at room temperature for two hours, remove the THF in vacuo, and add saturated aqueous NaHCO₃ until the mixture is neutral to hydrion paper. Extract the aqueous phase 3 times with CH₂Cl₂/i-PrOH (4:1) and dry the combined organic layers over Na₂SO₄. Dissolve the residue obtained after evaporation of solvent in 8 ml THF and, then, add 2 ml 1N HCl. Stir the mixture at room temperature for three hours and warm to 45° C. for 1.5 hours. Evaporation of solvent yields a crude solid. Purify this solid by silica gel radial chromatography eluting with a gradient of 3% to 6% 2N NH₃/MeOH in CH₂Cl₂. Dissolve the free base obtained after evaporation of solvent in a small amount of acetonitrile and add three drops 1N HCl. Evaporation of solvent yields the title product (0.054 g, 21% yield). Electrospray MS: 545 (M+1, parent ion).

### Preparation 20

### 2-Bromo-4-methanesulfonyl-1-methoxy-benzene

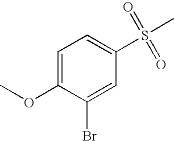

Dissolve 3-bromo-4-methoxybenzenesulfonyl chloride (2.86 g, 10 mmol) in 40 ml THF at room temperature. Add 10 ml of aqueous saturated ammonium chloride solution. While stirring, chill in an ice water bath and add zinc dust (0.72 g, 11 mmol) in small portions. Next add iodomethane dropwise over ten minutes and continue to stir the mixture in the ice bath for two hours. Partition the mixture between diethyl ether and water. Wash the organic layer with brine and dry over anhydrous sodium sulfate. Evaporate the solvent and recrystallize the crude solid from hexanes/diethyl ether to yield the title compound (0.34 g, 12% yield).

### EXAMPLE 10

### 9-Methanesulfonyl-5-[4-(2-piperidin-1-yl-ethoxy)-phenyl]-5H-6-oxa-chrysen-2-ol hydrochloride salt

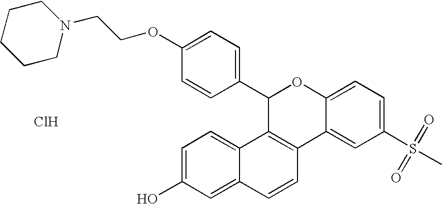

The title compound is prepared from 2-bromo-4-methanesulfonyl-1-methoxy-benzene and 2-trifluoromethanesulfonic acid 6-methoxy-1-[4-(2-piperidin-1-yl-ethoxy)-benzoyl]-napthalen-2-yl ester as described for the preparation of Examples 10 and 11. Electrospray MS: 530 (M+1, parent ion).

### Formulation

Because the free base form of a compound of formula I contains a basic moiety (i.e., amino), said compound may be formulated as a pharmaceutical acid addition salt, e.g., as the hydrochloride salt or as a salt described in “Handbook of Pharmaceutical Salts: Properties, Selection and Use”, Weinheim, New York: VHCA; Wiley-VCH, 2002.

The present pharmaceutical compositions are prepared by known procedures using well-known and readily available ingredients. In making the formulations of the present invention, the active ingredient (formula I compound) will usually be mixed with a carrier, or diluted by a carrier, or enclosed within a carrier which may be in the form of a capsule, sachet, paper or other container. When the carrier serves as a diluent, it may be a solid, semisolid or liquid material which acts as a vehicle, excipient or medium for the active ingredient.

Some examples of suitable carriers, excipients, and diluents include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water syrup, methyl cellulose, methyl and propylhydroxybenzoates, talc, magnesium stearate and mineral oil. The formulations can additionally include lubricating agents, wetting agents, emulsifying and suspending agents, preserving agents, sweetening agents or flavoring agents.

Biological Assays

Estrogen Receptor Binding Assay: Representative compounds of the present invention are screened for binding affinity to both estrogen receptor types (ERα and ERβ). This competition binding assay measures the compound's ability to displace ³H-estradiol and generates IC₅₀ and Kᵢ values for both receptor types.

This competition binding assay is run in a buffer containing 50 mM Hepes, pH 7.5, 1.5 mM EDTA, 150 mM NaCl, 10% glycerol, 1 mg/mL ovalbumin and 5 mM DTT, using 0.025 μCi per well ³H-Estradiol(NEN #NET517 at 118 Ci/mmol, 1 mCi/mL), 10 ng/well ERAlpha or ERbeta receptor (PanVera). A compound of the present invention is added at 10 different concentrations. Non-specific binding is determined in the presence of 1 μM of 17-B Estradiol. The binding reaction (140 μL) is incubated for 4 hours at room temperature, then 70 μl of cold DCC buffer is added to each reaction (DCC buffer contains per 50 mL of assay buffer, 750 mg of charcoal (Sigma) and 250 mg of dextran (Pharmacia)). Plates are mixed 8 minutes on an orbital shaker at 4° C. Plates are then centrifuged at 3,000 rpm at 4° C. for 10 minutes. An aliquot of 120 μl of the mix is transferred to another 96-well, white flat bottom plate (Costar) and 175 μl of Wallac Optiphase “Hisafe 3” scintillation fluid is added to each well. Plates are sealed and shaken vigorously on an orbital shaker. After an incubation of 2.5 hours, the plates are read in a Wallac Microbeta counter. The data is used to calculate an IC₅₀ and % Inhibition at 10 μM. The K_{d} for ³H-Estradiol is determined by saturation binding to ER alpha and ER beta receptors. The IC₅₀ values for test compounds are converted to Kᵢ using Cheng-Prusoff equation and the K_{d} determined by saturation binding assay.

Ishikawa Cell Proliferation Assay: This assay measures cell proliferation (using an alkaline phosphatase readout) in both an agonist mode in the presence of a compound of the present invention alone, and in an antagonist mode in which the ability of a compound of the present invention to block estradiol stimulation of growth is measured.

Ishikawa human endometrial tumor cells are maintained in MEM (minimum essential medium, with Earle's salts and L-Glutamine, Gibco BRL, Gaithersburg, Md.), supplemented with 10% fetal bovine serum (FBS) (V/V), (Gibco BRL). One day prior to assay, growth media is changed to assay medium, DMEM/F-12 (3:1) (Dulbecco's Modified Eagle Medium: Nutrient Mixture F-12, 3:1 Mixture, phenol red-free, Gibco BRL) supplemented with 5% dextran coated charcoal stripped fetal bovine serum (DCC-FBS) (Hyclone, Logen, Utah), L-Glutamine (2 mM), MEM sodium pyruvate (1 mM), HEPES (N-[2-hydroxyethyl]piperazine-N′-[2-ethanesulfonic acid] 2 mM) all from Gibco BRL). After an overnight incubation, Ishikawa cells are rinsed with Dulbecco's Phosphate Buffered Saline (1×) (D-PBS) without Ca⁺² and Mg⁺² (Gibco BRL), and trypsinized by a 3 minute incubation with 0.25% Trypsin/EDTA, phenol red-free (Gibco BRL). Cells are resuspended in assay medium and adjusted to 250,000 cells/mL. Approximately 25,000 cells in a 100 μl media are added to flat-bottom 96 wells microculture plates (Costar 3596) and incubated at 37° C. in a 5% CO₂ humidified incubator for 24 hours. The next day, serial dilutions of compounds are prepared in assay medium (at 6 times the final concentration in the assay). The assay is run in dual mode, agonist and antagonist modes.

For the agonist mode, plates receive 25 μl/well of assay medium followed by 25 μl/well of a diluted compound of the present invention (at 6× the final concentrations). For the antagonist mode, plates receive 25 μl/well of 6 nM E₂ (β-Estradiol, Sigma, St. Louis, Mo.) followed by 25 μl/well of a diluted compound of the present invention (at 6× the final concentrations). After an additional 48-hour incubation at 37° C. in a 5% CO₂ humidified incubator, media is aspirated from wells and 100 μl fresh assay medium is added to each microculture. Serial dilutions of compounds are prepared and added to the cells as described above. After an additional 72 hour incubation at 37° C. in a 5% CO₂ humidified incubator, the assay is quenched by removing media and rinsing plates twice in Dulbecco's Phosphate Buffered Saline (1×) (D-PBS) (Gibco BRL). The plates are dried for 5 minutes and frozen at −70° C. for at least 1 hour. The plates are then removed from the freezer and allowed to thaw at room temperature. To each well, 100 μl of 1-Step™ PNPP (Pierce Chemical Company, Rockford, Ill.) is added. After a 20-minute incubation, plates are read on a spectophotometer at 405 nm.

The data is fitted to a linear interpolation to derive EC₅₀ (for agonist mode) or IC₅₀ (for antagonist mode) values. For the antagonist mode, a % efficacy for each compound is calculated versus E2 (1 nM) alone. For the agonist mode, a % efficacy for each compound is calculated versus the response to tamoxifen.

In the agonist mode, the compounds of Examples 1, 3, 5, 7, 9, 10 and 11 were tested and were found to be less stimulatory than tamoxifen. For example, the compound of Example 1 had a relative % efficacy of 26%. In the antagonist mode, this same compound inhibited greater than at least 65% of the 1 nM estradiol response. For example, the compound of Example 1 had an IC₅₀ of 45 nM and a % efficacy of 92%.

MCF-7 Proliferation Assay: The MCF-7 cell line is derived from a human breast adenocarcinoma and is used as an indicator of potential antiproliferative activity in breast epithelium.

MCF-7 breast adenocarcinoma cells (ATCC HTB 22) are maintained in MEM (minimal essential medium, phenol red-free, Gibco BRL) supplemented with 10% fetal bovine serum (FBS) (V/V), L-glutamine (2 mM), sodium pyruvate (1 mM), HEPES ((N-[2-hydroxyethyl]piperazine-N′-[2-ethanesulfonic acid]10 mM}, non-essential amino acids (0.1 mM) and Penicillin Streptomycin (1×). Seven days prior to assay, MCF-7 cells are switched to assay media which is the same as maintenance medium except supplemented with 10% dextran-coated charcoal-stripped fetal bovine serum (DCC-FBS) assay medium in place of 10% FBS. MCF-7 cells are removed from flasks using 10×Trypsin EDTA (phenol red free, Gibco BRL) and diluted to 1× in (Ca++/Mg++ free HBSS (phenol red-free). Cells are adjusted to 80,000 cells/mL in assay medium. Approximately 8,000 cells (100 μl) are added to each well in 96 well Cytostar T scintillation plates (Amersham) and incubated at 37° C. in a 5% CO₂ humidified incubator for 24 hours to allow cell adherence and equilibration after transfer.

Serial dilutions of a compound of the present invention are prepared in assay medium at 4× the final desired concentration). A 50 μl aliquot of test compound dilutions (at 4× the final assay concentration) is transferred to duplicate wells followed by 50 μl assay medium for the agonist mode or 50 μl of 40 pM of E2 for the antagonist mode to a final volume of 200 μl. For each of the agonist plates, a basal level (media) and a maximum stimulated level (with 1 μM E2) is determined. For each of the antagonist plates, a basal level (media) and an E2 (10 pM) alone control is determined. After an additional 48 hours at 37° C. in a 5% CO₂ humidified incubator, 20 μl of assay medium containing 0.01 μCi of ¹⁴C-thymidine (52 mCi/mmol, 50 μCi/ul, Amersham) is added to each well. The plates are incubated overnight in the same incubator and then counted on the Wallac Microbeta counter. The data is averaged to calculate an IC₅₀ and % inhibition@1 μM for the antagonist mode. For the agonist mode, an EC₅₀ and percent of maximum E2 stimulation and concentration of maximum stimulation is calculated.

3-Day Rat Uterus Antagonist Assay: This model for uterine antagonism utilizes immature (3 week old) female rats that are highly sensitive to estrogenic stimulation of the uterus given that their circulating estrogen levels are prepubertal. The uteri from immature rats are fully responsive to exogenous estrogen, yet are quiescent in the absence of exogenous estrogen. Administration of exogenous estrogen to immature rats produces a reliable elevation of uterine weight, which can be used to study uterine antagonist effects. The rats are treated with both estradiol and 4 different concentrations of a compound of the present invention for 3 days and then uterine wet weights are measured.

Nineteen to twenty-one day old (or 45-50 g) female rats are orally treated with E2 (0.1 mg/kg, a maximal stimulatory estrogenic stimulus for reliably increasing uterine weight) and 10, 1.0, 0.1 and 0.01 mg/kg test compound for 3 days, 6 rats per group. Test compounds are dissolved in 20% β-hydroxycyclodextrin and administered by oral gavage in a volume of 0.2 mL daily (15 min. prior to the ethynyl estradiol gavage). A vehicle control, E2 alone and E2+raloxifene are also done as controls. The animals are fasted overnight following the final dose. On the following morning, the animals are weighed, then euthanized (by carbon dioxide asphyxiation) and the uteri rapidly collected (via a mid-line ventral incision) and weighed.

Uterine weight/body weight ratios (UWR) are calculated for each animal. The percent inhibition of the estrogen-induced response is then calculated by the following formula: percent inhibition=100×(UWR_{estrogen}−UWR_{test compound}/UWR_{estrogen}−UWR_{control}). ED₅₀ values are derived from a semi-log regression analysis of the linear aspect of the dose response curve. Both the UWR data and the percent inhibition data are statistically analyzed by one way analysis of variance (ANOVA) with post-hoc testing by Fisher's PLSD when indicated by a p≦0.05. Statistical analyses are performed using the Statview® 4.0 software package.

The compounds of Examples 1 and 3 were tested in the above assay and were found to inhibit the estrogen-induced response when administered at 0.01, 0.1 and 1.0 mg/kg. For example, the compounds of Examples 1 and 3 had an ED₅₀ of 0.22 and 0.17 mpk and a % antagonism of 69 and 81%, respectively.

4-Day OVX Rat Uterine Agonist Assay: In order to assure that a test compound does not have any partial uterine agonist activity, compounds are administered to mature, ovariectomized rats.

Seventy-five day old rats are ovariectomized and treatment is started 14 days later when circulating estradiol levels have reached minimal levels. After 4 days of treatment with 3 doses of a compound of the present invention, (6 rats per group) body weight, uterine wet weight and uterine eosinophil peroxidase (EPO) activity are measured. Cholesterol levels are also measured to compare relative ability to lower cholesterol with other SERMs. If there is any question of uterine stimulation, histological examination will determine epithelial cell height.

10-Day Rat Hormone (Ovarian Stimulation) Screen: An initial, first screen for ovarian toxicity is conducted using a 10-day rat hormone study to measure estradiol and luteinizing hormone levels after compound administration. This screen is conducted by administering compound by oral gavage for 10 days to mature (9-10 week old) F344 female rats. Trunk blood is collected by rapid decapitation for evaluation of LH and estradiol levels approximately 2 hours after the 10^{th} dose. Serum, obtained by centrifugation, is removed and stored frozen below −60° C. until assayed. Serum levels of LH and estradiol are measured using radioimmunoassay (RIA) methods.

Rat LH primary antibody and reference preparations (rat LH:RP-3) are obtained from Dr. A. F. Parlow, Director, Pituitary Hormones and Antisera Center, Harbor-UCLA Medical Center, Torrance, Calif. The LH assay upper limits of detection are 30 ng/mL and the lower limits of detection are 0.1 ng/mL for the 100 μl samples.

E2 Clinical Assays. DiaSorin s.r.l., Saluggia (Vercelli), Italy. The upper limit of detection is 1000 pg/mL and the lower limit of detection is 5 pg/mL. The compound of Example 3 was tested in the above assay and did not significantly elevate circulating estradiol or LH levels.

35-Day Ovary-Intact Rat Bone Assay: While previous SERMs, including raloxifene have shown efficacy in preventing bone loss in OVX rats, the possibility of interference with estrogen-regulated turnover in ovary-intact rats needs to be addressed.

This assay is done in mature rats with concentrations based on the demonstrated efficacy in the 3-day assay. Generally, at least three concentrations are chosen based on multiples of the ED₅₀ generated therein. These multiples are generally 1×, 10× and 30× the ED₅₀. A compound of the present invention is administered to an OVX rat for 35 days and is compared to control, ovariectomized, and/or GnRH-administered rats. Femurs, tibiae, uteri, ovaries and serum are taken for further analyses. DEXA (Dual Energy X-ray Absorptivity), CT (Computed Tomography) and histologic analysis are done on the long bones to assess any changes. CT scans of the distal femur are done to calculate BMD (bone mineral density), cross sectional area and BMC (bone mineral content). Bone strength measurements (load to failure) may also be done to determine consequences of any bone mass or material changes. Uterine and ovarian histology are examined to confirm long term dosing effects of uterine efficacy and potential ovarian stimulation. The serum is analyzed for LH and E2 levels as a possible indicator of ovarian effects.

### Utilities

The diseases, disorders or conditions for which a compound of formula I is useful in treating include, but are not limited to, (1) uterine cancer; (2) endometriosis; (3) uterine leiomyoma/leiomyomata; (4) post-menopausal osteoporosis, i.e., osteoporosis caused by the loss of bone that results from a lack of endogenous estrogen such as occurs in a woman following cessation of menstration due to natural, surgical, or other processes; and (5) estrogen receptor positive (ER+) breast cancer, particularly the prevention thereof. Treatment of uterine leiomyoma/leiomyomata as described herein, also contemplates the reduction of the occurrence or severity of the associated symptoms such as pain, urinary frequency, and uterine bleeding.

### Dose

The specific dose administered is determined by the particular circumstances surrounding each situation. These circumstances include, the route of administration, the prior medical history of the recipient, the pathological condition or symptom being treated, the severity of the condition/symptom being treated, and the age of the recipient. The recipient patient's physician should determine the therapeutic dose administered in light of the relevant circumstances.

Generally, an effective minimum daily dose of a compound of formula I will exceed about 5 mg. Typically, an effective maximum daily dose will not exceed about 350 mg. The exact dose may be determined, in accordance with the standard practice in the medical arts of “dose titrating” the recipient; that is, initially administering a low dose of the compound, and gradually increasing the does until the desired therapeutic effect is observed.

1. A compound of formula I: 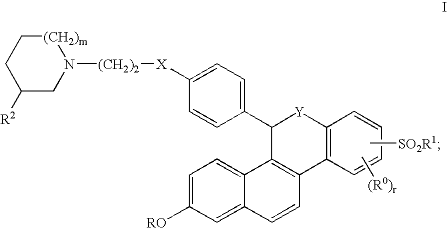
wherein:
m and r are independently 0, 1 or 2;
R is H, SO₂(n-C₄-C₆ alkyl) or COR³;
R⁰ is independently at each occurrence OH, CF₃, halo, C₁-C₆ alkyl or C₁-C₆ alkoxy;
R¹ is C₁-C₆ alkyl, C₁-C₆ alkoxy, NR⁴R^{4a}, CF₃ or CH₂CF₃;
R² is H or methyl provided that if m is 1 or 2, then R² must be H and that if m is 0, then R² must be methyl;
R³ is C₁-C₆ alkyl, C₁-C₆ alkoxy, NR⁶R^{6a}, phenoxy, or phenyl optionally substituted with halo;
R⁴ is C₁-C₆ alkyl or phenyl;
R^{4a}, R⁶ and R^{6a} are independently at each occurrence H, C₁-C₆ alkyl or phenyl;
X is O or NR⁷;
Y is O or S; and
R⁷ is H or C₁-C₆ alkyl; or a pharmaceutical acid addition salt thereof.

2. The compound of claim 1 wherein X and Y are O and m is 1 or 2.

3. The compound of claim 2 wherein r is 0.

4. The compound of claim 3 wherein R is H or COR³ and R³ is C₁-C₄ alkyl, NHCH₃ or phenyl.

5. The compound of claim 4 wherein R is H and m is 1.

6. The compound of claim 5 wherein the SO₂R¹ moiety is at the 4-position.

7. The compound of claim 6 wherein R¹ is C₁-C₄ alkyl, CF₃ or NR⁴R^{4a} and R⁴ is C₁-C₄ alkyl and R^{4a} is H or C₁-C₄ alkyl.

8. The compound of claim 7 wherein R¹ is methyl, ethyl, cyclopropyl, CF₃, NHCH₃ or N(CH₃)₂.

9. The compound of claim 1 selected from the group consisting of: 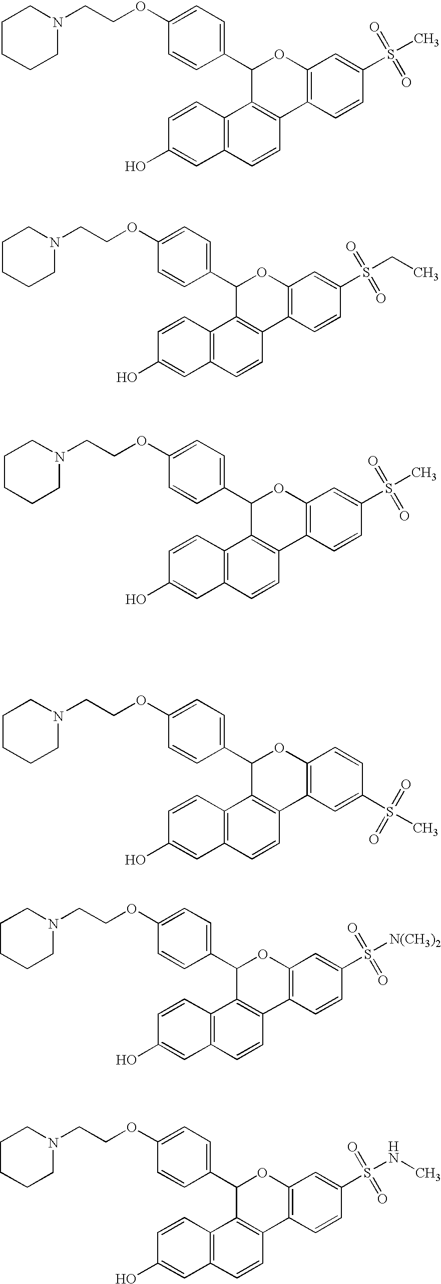 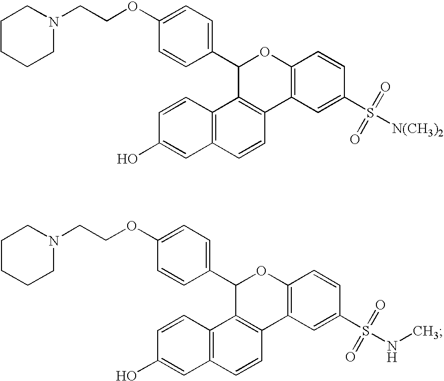
or a pharmaceutical acid addition salt thereof.

10. (canceled)

11. (canceled)

12. A method of treating uterine leiomyoma comprising administering to a patient in need thereof an effective amount of a compound of claim 1.

13. (canceled)

14. A compound of formula II: 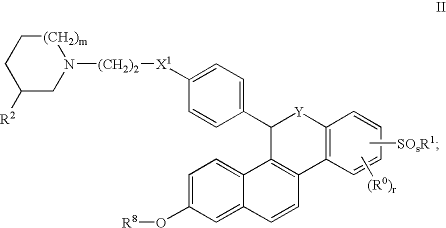
wherein:
m and r are independently 0, 1 or 2;
q is 0 or 1;
s is 0, 1 or 2;
R⁰ is independently at each occurrence OH, CF₃, halo, C₁-C₆ alkyl or C₁-C₆ alkoxy;
R¹ is C₁-C₆ alkyl, C₁-C₆ alkoxy, NR⁴R^{4a}, CF₃ or CH₂CF₃;
R² is H or methyl provided that if m is 1 or 2, then R² must be H and that if m is 0, then R² must be methyl;
R⁸ is H, C₁-C₆ alkyl, benzyl, SO₂CH₃, SO₂(n-C₄-C₆ alkyl) or COR³;
R³ is C₁-C₆ alkyl, C₁-C₆ alkoxy, NR⁶R⁶a, phenoxy, or phenyl optionally substituted with halo;
R⁴ is C₁-C₆ alkyl or phenyl;
R^{4a}, R⁶ and R^{6a} are independently at each occurrence H, C₁-C₆ alkyl or phenyl;
X¹ is O or NR⁹;
Y is O or S; and
R⁹ is H, C₁-C₆ alkyl or CO₂(C₁-C₆ alkyl); provided that if s is 2, then R⁸ is C₁-C₆ alkyl, SO₂CH₃ or benzyl or R⁹ is CO₂(C₁-C₆ alkyl); or an acid addition salt thereof.

15. The compound of claim 14 wherein X¹ and Y are O and m is 1 or 2.

16. The compound of claim 15 wherein r is 0.

17. The compound of claim 16 wherein R⁸ is SO₂CH₃, benzyl or methyl.

18. The compound of claim 17 wherein m is 1.

19. The compound of claim 18 wherein the SO_{S}R¹ moiety is at the 4-position.

20. The compound of claim 19 wherein R¹ is C₁-C₄ alkyl, CF₃ or NR⁴R^{4a} and R⁴ is C₁-C₄ alkyl and R^{4a} is H or C₁-C₄ alkyl.

21. The compound of claim 20 wherein R¹ is methyl, ethyl, cyclopropyl, CF₃, NHCH₃ or N(CH₃)₂.
